(19) United States
(12) Patent Application Publication
(10) Pub. No.: US 2010/0058654 A1
(43) Pub. Date: Mar. 11, 2010

(54) Catalytic conversion of liquid alcohols to liquid ethers and oxygenated gasoline

(75) Inventor: Melvin Keith Carter, Lincoln, CA (US)

Correspondence Address: Carter Technologies, 2300 Sutter View Lane, Lincoln, CA, 95648, US

(73) Assignee: Carter Technologies, Lincoln, CA (US)

(21) Appl. No.: 12/231,592

(22) Filed: Sep. 5, 2008

(51) Int. Cl. C10L 1/185; C07C 41/01

(52) U.S. Cl. 44/447; 568/671

(57) ABSTRACT

Catalyst based reactions are taught for non-oxidative chemical conversion of liquid alcohols to higher boiling alcohols, ethers, glycol ethers, amines and oxygenated gasoline products, comprising ethanol to butanol, propanols to hexanols, butanols to octanols, ethanol plus butanol to ethyl butyl ethers, ethanol plus ethyl butyl ethers to ethyl hexyl ethers as oxygenated gasoline, ethanol plus ethyl hexyl ethers to butyl hexyl ethers as oxygenated gasoline, n-butyl amine plus ethanol to ethyl butyl amine, hexyl amines and related products at ambient pressure. This same catalytic chemistry also converts substituted liquid organic compounds comprising aldehydes, ketones, ethers, esters, organic acids and thiols possessing at least one active hydrogen to related alkylated organic products in the absence of air.

Catalysts are based on selected transition metal complexes possessing a degree of symmetry. Laboratory results have demonstrated [chromium(II)]₂, [cobalt(II)]₂, [vanadium(II)]₂ and similar families of catalysts to be effective for non-oxidative catalytic conversion of substituted organic compounds to products comprising related higher molecular weight compounds in good yields in the absence of air, at modest temperatures and pressures.

### REFERENCES CITED

U.S. Patent Documents

| Patent No. | Issue Date | Author | Comments |
|---|---|---|---|
| 6,350,918 | Feb. 26, 2002 | IE Wachs & Y Cai | Aldehyde from alcohol at 150 C-600 C over oxide of V, Cr, |
| | | | Mo, W, Re |
| 6,348,619 | Feb. 19, 2002 | Y. Yoshida, Y. Mikami | Ester from an aldehyde + alcohol + O₂ at 0-100 C over |
| | | and M. Oh-Kita | Pd—Bi—Pb catalyst |
| 6,166,265 | Dec. 26, 2000 | J Kanand & R Paciello | Buteraldehyde + Butanol from butadiene + ROH > latm P |
| | | | on acid or transition metal catalyst |
| 6,107,526 | Aug. 22, 2000 | S. J. Frey, S. P. Davis, S. I. Krupa | t-BuOEt from ethanol + iso-butene/cat. at 30-75 C/65-185 psi |
| | | & P. R. Cottrell | |
| 5,986,138 | Nov. 16, 1999 | B. Satyavathi, A. N. R. Patwari | φNHR from φNH₂ + EtOH at 300°-400° C. over FeOy/TiO₂ |
| | | & U. T. Bhalerao | or others |

### BACKGROUND

1. Field of the Invention

This invention relates to ambient pressure non-oxidative catalytic chemical combination or alkylation in a liquid range of an organic reactant comprising an alcohol, glycol, aldehydes, ketones, ethers, esters, organic acids, alkanes, alkenes, amines, imines, thiols, sulfides, phosphines and other substituted organic compound, possessing at least one active hydrogen atom, with another organic reactant forming products including oxygenated gasoline. Specifically, this application discloses efficient catalytic conversion of chemical compounds produced from renewable resources including liquid ethanol and/or other available compounds to higher boiling alcohols, ethers, glycol ethers, oxygenated gasoline, oils, polymers or other products as listed above in the absence of air at reflux temperatures employing catalysts based on transition metal complexes of low valence possessing a degree of symmetry as described herein.

2. Description of Prior Art

The chemical process industry has grown to maturity based on petroleum feed stocks. Petroleum is a non-renewable resource that may become unavailable in the next 100 to 150 years. This planet Earth fosters continual growth of numerous carbohydrate based plants including fruits, vegetables and grain food sources plus their supporting plant stalks and related natural waste materials for recycle. Grains, corn cobs, the support plant stalks and certain grasses are, in part, subject to direct chemical conversion and bio-fermentation processes producing ethanol and related products. A major industry is blooming in ethanol production by conversion of cellulose materials where much of the product is sold as combustion engine fuel or its additive. Ethanol is becoming more available as a renewable resource and this application teaches its catalytic conversion to valuable oxygenated gasoline and chemical intermediates for use in the automotive fuels and chemical process industries.

A number of chemical reaction paths have previously been taught for conversion of aliphatic alcohols to higher molecular weight alcohols and related products using carbonyl insertion, partial oxidation and other gas phase processes but do not teach high conversion efficiencies in liquid form without employment of high temperature and pressure, aggressive chemical oxidizers, mineral acids or strong chemical agents. Gaseous ethanol has been converted to ethyl ether at 120° C. and to ethylene at 180° C. over a dehydrating acid. Controlled oxidation of methane at high temperatures, previously investigated under a wide range of conditions, has produced carbon dioxide, carbon monoxide, low concentrations of unsaturated hydrocarbons, oligomers, low levels of alcohols, aldehydes and water. However, these efforts have not produced significant amounts of aldehydes or alcohols. As a result direct conversion of saturated hydrocarbons to aldehydes and/or alcohols has essentially been abandoned in favor of conversion of more labile hydrocarbons such as alkenes, vinyl alcohols or other organic compounds having reactive groups. Secondary and tertiary alcohols can be produced from branched olefins in the gas phase by combining with water or primary alcohols in the presence of sulfuric acid. Higher boiling branched alcohols have been produced from primary alcohols over sodium alkoxide in the presence of a nickel catalyst at 200° C. to 250° C. in the gas phase by means of the Guerbet reaction. Alcohols can also be dehydrated to form olefins over aluminum oxide at 350° C. to 450° C. or by means of other conditions in the gas phase, often at elevated pressure.

There are several other hot tube reactions described in the scientific and patent literature for conversion of gaseous alcohols to a wide range of low concentration products from gasoline type hydrocarbons to aldehydes and ethers. Aldehydes and ketones can be formed by passing alcohol vapors over Cu and its alloys or Ag at 300° C. to 600° C. in the presence of controlled amounts of air. U.S. Pat. No. 6,166,265, issued Dec. 26, 2000, introduced a process for preparation of n-butyraldehyde and/or n-butanol by reacting butadiene with vaporized alcohol at super-atmospheric pressure and elevated temperatures using an acid resin or one of several transition metal oxides. U.S. Pat. No. 6,350,918, issued Feb. 26, 2002, teaches a process for the selective oxidation of alcohols to aldehydes in the vapor phase at 150° C. to 600° C. over oxides of V, Cr, Mo, W or Re in their high oxidation states. Less selective chemistry may oxidize vaporized alcohol to aldehydes and ketones. Aldehydes can also be produced by a chemical exchange where one oxidized organic compound may transfer its oxygen atoms to an alcohol converting it to an aldehyde.

Alkyl ethers are commonly produced from branched hydrocarbons in a distillation process at elevated temperature and pressure. Alkyl tertiary butyl ethers have been produced in this manner for application as gasoline additives. U.S. Pat. No. 6,107,526, issued Aug. 22, 2000, disclosed addition of ethanol to iso-butene (from dehydration of iso-butane) in contact with a catalyst at 65 to 185 pounds per square inch pressure and 30° C. to 75° C. in formation of ethyl tertiary butyl ether during a distillation process.

Catalytic chemistries have also been taught in the production of other products. Vapor phase alkylation of aromatic amines over an iron oxide/titanium oxide catalyst performed at 300° C. to 400° C. was reported in U.S. Pat. No. 5,986,138, issued Nov. 16, 1999. U.S. Pat. No. 6,348,619, issued Feb. 19, 2002, disclosed the formation of esters wherein oxygen or air is passed through an alcohol plus a selected aldehyde at 0° C. to 100° C. over a palladium-bismuth-lead catalyst.

The above reported chemistries have been conducted primarily in the gas phase, in the presence of oxygen or air and/or under high pressure and are, therefore, distinctly different from catalytic conversions conducted in the absence of air or oxygen, in the liquid phase as taught herein. The invention disclosed in this application teaches non-oxidative catalytic conversion of liquid alcohols, glycols, aldehydes, ketones, ethers, esters, organic acids, amines, thiols and phosphines possessing at least one active hydrogen to related alkylated organic products including oxygenated gasoline in the absence of air using mono-metal, di-metal, tri-metal and/or poly-metal backbone or molecular string type transition metal catalysts in a low oxidation state without addition of aggressive chemical oxidizing agents and without addition of other strong chemicals. In addition, amines, diamines and polyamines may be alkylated and/or converted to similar higher molecular weight products, thiols, dithiols and polythiols can be alkylated and/or converted to similar higher molecular weight products and phosphines, diphosphines and polyphosphines may also be alkalyted and/or converted to similar higher molecular weight products. It also discloses non-oxidative catalytic conversion of liquid alcohols, amines, aldehydes, and/or any organic compounds possessing reactive hydrogen atoms to higher molecular weight compounds by means of alkylation chemistry. No labile or other reactive chemical groups are required, although drying agents may be employed for removal of water or other by products. Use of selected mono-metal, di-metal, tri-metal and/or poly-metal backbone or molecular string type transition metal catalysts produced in the absence of air, described in this application, result in high yields of the reported products.

### SUMMARY OF THE INVENTION

This invention describes non-oxidative catalytic chemical methods using selected members of transition metal catalysts in their low valence states, possessing a high degree of symmetry, for conversion of liquid alcohols to higher boiling alcohols, ethers, glycol ethers, amines and oxygenated gasoline products, comprising ethanol to butanol, propanols to hexanols, butanols to octanols, ethanol plus butanol to ethyl butyl ethers, ethanol plus ethyl butyl ethers to ethyl hexyl ethers as oxygenated gasoline, ethanol plus ethyl hexyl ethers to butyl hexyl ethers as oxygenated gasoline, n-butyl amine plus ethanol to ethyl butyl amine, hexyl amines and related products at ambient pressure. This same catalytic chemistry also converts substituted organic compounds comprising aldehydes, ketones, ethers, esters, organic acids, thiols, sulfides and phosphines possessing at least one active hydrogen to related alkylated organic products in the absence of air. This catalytic chemical conversion method operates throughout the liquidus range of the reactants in the absence of air at ambient pressure. It is an object of this invention, therefore, to provide a non-oxidative mono-metal or string transition metal catalytic process facilitating conversion of alcohols in a liquid state to higher molecular weight alcohols, ethers, glycols and related products in the absence of air at ambient pressure. It is another object of this invention to catalytically alkylate ketones to higher molecular weight ketones, allenyl ethers and similar products in the absence of air at ambient pressure. It is still another object of this invention to catalytically alkylate amines to higher molecular weight amine products in the absence of air at ambient pressure. It is still another object of this invention to catalytically convert liquid ethanol to oxygenated gasoline at reflux temperatures.

Other objects of this invention will be apparent from the detailed description thereof which follows, and from the claims.

### DETAILED DESCRIPTION OF THE INVENTION

A process for non-oxidative chemical conversion of liquid alcohols to higher boiling alcohols, ethers, glycol ethers, amines and oxygenated gasoline products, comprising ethanol to butanol, propanols to hexanols, butanols to octanols, ethanol plus butanol to ethyl butyl ethers, ethanol plus ethyl butyl ethers to ethyl hexyl ethers as oxygenated gasoline, ethanol plus ethyl hexyl ethers to butyl hexyl ethers as oxygenated gasoline, n-butyl amine plus ethanol to ethyl butyl amine, hexyl amines and related products at ambient pressure. This same catalytic chemistry also converts substituted liquid organic compounds comprising aldehydes, ketones, ethers, esters, organic acids and thiols possessing at least one active hydrogen to related alkylated organic products in the absence of air. This process employs transition metal compounds, comprising [vanadium]₂, [chromium]₂ or [cobalt]₂ type compounds, for which the transition metals and directly attached atoms possess C₄ᵥ, D₄ₕ or D_{2d} point group symmetry. These catalysts have been designed based on a formal theory of catalysis, and the catalysts have been produced, and tested to prove their activity. The theory of catalysis rests upon a requirement that a catalyst possess a single metal atom or a molecular string such that transitions from one molecular electronic configuration to another be barrier free so reactants may proceed freely to products as driven by thermodynamic considerations. Catalysts effective for chemical conversion of liquid alcohols to products can be made from mono-metal, di-metal, tri-metal and/or poly-metal backbone or molecular string type compounds of the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof. These catalysts are made in the absence of oxygen so as to produce compounds wherein the oxidation state of the transition metal is low, typically monovalent or divalent although zero valent metal catalysts may also be produced. Anions employed for these catalysts comprise fluoride, chloride, bromide, iodide, cyanide, isocyanate, thiocyanate, sulfate, phosphate, oxide, hydroxide, oxalate, acetate, organic chelating agents and/or other groups. Mixed transition metal compounds have also been found to be effective catalysts for non-oxidative chemical conversions.

These catalysts act on alcohols, glycols, aldehydes, ketones, ethers, esters, organic acids, amines, thiols, sulfides, phosphines and most organic compounds possessing at least one active hydrogen to generate free radicals in times believed to be the order of or less than that of a normal molecular vibration. This may be viewed as generation of free radicals in equilibrium as indicated hereinafter, namely CH₃CH₂OH⇄CH₃CH₂•+•OH, CH₃CH₂OH⇄CH₃CH₂O•+•H and related for alkylation of and alkyl ether addition to available organic reactants. Catalytic exposure causes ethanol and other polar organic compounds to become alkylating agents provided water or other condensate by products formed are removed from the reaction sphere. Thus, ethanol, the exemplary compound applied throughout this application, reacts with itself to produce butanol, ethyl butyl ether and similar higher molecular weight compounds plus water wherein catalytically generated alkyl radicals attack both ethyl and hydroxyl sites, and the water so formed is removed by a dehydrating agent such as lime or dehydrated calcium sulfate.

Ethanol mixed in roughly equal molar concentrations with other compounds can alkylate them and produce a wide range of products. Ethanol reacts with itself or normal butanol in the presence of a selected catalyst to produce ethyl butyl ether, ethyl hexyl ether, di-butyl ethers and oxygenated gasoline products plus water. Ethanol can react with a ketone such as acetone to form 2-pentanone, an allyl ether and related products plus water. Ethanol plus normal butyl amine produces ethyl butyl amine, ethyl hexyl amines, di-butyl amines and related products plus water. Ethanol plus normal butyl thiol produces ethyl butyl sulfide, ethyl hexyl sulfides, di-butyl sulfides and other products plus water. Ethanol plus normal butyl phosphine produces ethyl butyl phosphines, ethyl hexyl phosphines, di-butyl phosphines and other products plus water.

Ethanol can selectively alkylate an alkene to preserve the double bond or add across it. For example, ethanol mixed with cyclohexene may form ethyl cyclohexene and ethyl cyclohexane products. Ethanol plus phenol may produce ethyl phenyl ether and related compounds. Thus, polar compounds activated by the selected catalysts taught herein produce alkylating agents available to those compounds present in the reaction vessel.

### Catalyst Selection Considerations

A Concepts of Catalysis effort formed a basis for selecting molecular catalysts for specified chemical reactions through computational methods by means of the following six process steps. An acceptable chemical conversion mechanism, involving a single or pair of transition metal atoms, was established for the reactants (step 1). A specific transition metal, such as cobalt, was selected as a possible catalytic site as found in an M or M-M string (step 2), bonded with reactant molecules in essentially a C₄ᵥ, D_{2d} or D₄ₕ point group symmetry configuration, and having a computed bonding energy to the associated reactants of −5 to −60 kcal/mol (step 3). The first valence state for which the energy values were two-fold degenerate was 2+ in most cases although 1+ is possible (step 4). Cyanide, chloride and other anions may be chosen provided they are chemically compatible with the metal in formation of the catalyst (step 5). An inspection should also be conducted to establish compliance with the rule of 18 (or 32) to stabilize the catalyst; thus, compatible ligands may be added to complete the coordination shell (step 6). This same process may be applied for selection of a catalyst using any of the first, second or third row transition metals, however, only those with acceptable negative bonding energies can produce effective catalysts. The approximate relative bonding energy values may be computed using a semi-empirical algorithm or other means. Such a computational method indicated that any of the first row transition metal complexes may be anticipated to produce usable catalysts once the outer coordination shell had been completed with ligands, even though only the elements Ti, Cr, Mn, Co, Ni and Cu indicated reasonable bonding energies for the first row transition metals in a simplified molecular model. In general, preliminary energy values computed for transition metal ethanol complexes were indicated to produce useable catalysts once bonding ligands have been added.

Catalyst structures commonly including a pair of bonded transition metal atoms require chelating ligands and/or bonding orbital structures that may be different for each metal. The following compounds comprise a limited selection of examples. For the first row transition metals vanadium catalysts comprise vanadium(II) oxide, (VO)₂, and (VF₂)₂ having V—V bonds and ethylenediamine (EDA) links the metals in (VCl₂)₂EDA₂ while ethanol or other reactants may displace a CO and/or a THF in the compound [V(THF)₄Cl₂][V(CO)₆]₂. Chromium catalysts comprise Cr(O₂CCH₃)₂(HO₂CCH₃)₂, Cr₂[CH₃(C₅H₃N)O]₄, (CrCl₂)₂•2EDA, (CrBr₂)₂EDA₂, [Cr(OH)₂]₂EDA₂ and Cr₂(O₂CCH₃)₄(H₂O)₂ where a reactant may displace waters of hydration. Manganese catalysts comprise [Mn(diethyldithiocarbamate)]ₙ, (MnCl₂)₂EDA₂, K₂[Mn₂Cl₆(H₂O)₄] and Mn₂(C₅H₈O₂)₄(H₂O)₂. Iron catalysts comprise (FeCl₂)₂EDA₂ and (FeBr₂)₂EDA₂. Cobalt cataysts comprise Co₂(C₆H₅O₂)₂(C₆H₆O₂)₂, Co₂, (C₅H₈O₂)₄(H₂O)₂, Co(C₆H₅O₂)₂(C₆H₆O₂)₂, Co₂C₆H₅O₂)₄, Ca₃[Co₂(CN)₁₀]13H₂O and [Co(CN)₂]₂K₃Cu(CN)₄. Nickel catalysts comprise Ni₂(C₆H₅N₃C₆H₅), Ni₂Br₂(C₈H₆N₂) and Ni₂S₂(C₂H₂C₆H₅). Copper catalysts comprise [CuO₂CC₆H₅]₄, [CuO₂CCH₃]₄, (CuCl)₂(EtOH)₄, (CuCN)₂(EtOH)₄ and K₂Cu₄(μ₂SC₆H₅)₆.

Second and third row transition metals are organized in groups or pairs. Zirconium, hafnium, nobelium and tantalum comprise (ZrCl₂)₂, (HfCl₂)₂, (HfF₂)₂, (NbCl₂)₂, (TaCl₂)₂ and (TaF₂)₂. Molybdenum and tungsten catalysts comprise [Mo(CO)₄Cl₂]₂, [W(CO)₄Cl₂]₂, [K₄MoCl₆]₂, [Mo(CN)₂]₂K₃Cu(CN)₄, [W(CN)₂]₂K₃Cu(CN)₄, [Mo(Cl)₂]₂K₃Cu(CN)₄ and [W(Cl)₂]₂K₃Cu(CN)₄. Rhenium and technetium catalysts comprise [Re(CO)₂Cl₂(PR₃)₃]₂ and [Tc(CO)₂Cl₂(PR₃)₃]₂. Platinum, palladium, ruthenium, rhodium, osmium and iridium catalysts comprise (PtF₂)₂, (PdF₂)₂, [RuCl₂]₂EDA₄, [RhCl₂]₂EDA₄, [Ru(C₈H₆N₂)₂Cl₂]₂, [Rh(C₈H₆N₂)₂Cl₂]₂, Ru₂(O₂CR)₄Cl, Rh₂(O₂CR)₄Cl, [PdCl₄(PBu₃)₂]₂, [PtCl₄(PBu₃)₂]₂, [OsCl₂]₂EDA₄ and [IrCl₂]₂EDA₄. Silver and gold catalysts comprise (AgCN)₂K₃Cu(CN)₄ and (AuCN)₂K₃Cu(CN)₄.

A select number of single transition metal atom catalyst complexes containing four ligands each belong to the required point group symmetry. These catalysts comprise M(II)(C₆H₅O₂)₂(C₆H₆O₂)₂, M(II)(p-C₆H₅O₂)₂, M(II)(C₆H₆NO)₂(C₆H₇NO)₂ and M(II)(O₂CCH₃) ₂(HO₂CCH₃)₂ plus possible solvation ligands where M represents titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum or gold. In a limited number of complexes the transition metal atom may be monovalent.

### Description of Catalyst Preparation and Chemical Conversion

Catalyst preparation was conducted using nitrogen purging and/or nitrogen blanketing to minimize or eliminate air oxidation of the transition metal compounds during preparation. Transition metal catalysts, effective for ambient pressure conversion of substituted organic compounds, can be produced by combining transition metal salts in their lowest standard oxidation states with other reactants. Thus, such transition metal catalysts can be made by partially reacting transition metal (I or II) chlorides, bromides, sulfates, cyanides or similar compounds with transition metal (I or II) compounds and chelates or by forming transition metal compounds in a reduced state by similar means where mono-, di-, tri- and/or poly-metal compounds result. Some examples follow.

### EXAMPLE 1

The Co₂(C₆H₅O₂)₄ catalyst was prepared in a nitrogen atmosphere by addition of 0.660 grams (6 mmol) of pyrocatechol dissolved in 3.5 mL of nitrogen purged water to 0.7138 grams (3 mmol) of cobalt (II) chloride hexahydrate dissolved in 3 mL of nitrogen purged water with mixing and addition of 2N sodium hydroxide drop wise to attain a pH of 7. An insoluble dark green to black solid product formed. The suspended catalyst was used as prepared.

### EXAMPLE 2

The Co(O₂CCH₃)₂(NH₄O₂CCH₃)₂ catalyst was prepared in a nitrogen atmosphere by addition of 0.154 grams (2 mmol) of ammonium acetate to 0.250 grams (1 mmol) of light pink colored cobalt (II) acetate tetrahydrate dispersed in 4 mL of nitrogen purged ethanol with mixing. A soluble deep magenta to purple product solution formed. The dissolved catalyst was used as prepared.

### EXAMPLE 3

Preparation of the Cr₂(O₂CCH₃)₄ catalyst was conducted under nitrogen by reduction of 5.06 grams (19 mmol) of CrCl₃•6H2₂O dissolved in 15 mL of dilute hydrochloric acid by slow addition of approximately 7 grams of zinc dust followed by addition to 2.93 grams (38 mmol) of ammonium acetate dissolved in 35 mL of water with mixing. A purple colored solution of the catalyst formed.

### EXAMPLE 4

The compound V₂(O₂CCH₃)₄ was prepared as described by dispersing 1.82 grams of vanadium pentoxide in 10 grams of pure water, dissolving 3.08 grams of ammonium acetate and 4.48 grams of concentrated hydrochloric acid. This liquid was gently purged with nitrogen gas to displace dissolved oxygen and 6.5 grams of zinc dust was added in portions during a 5 minute period. The red brown dispersion changed to a pale blue colored solution as the catalyst formed.

Organic chemical conversions were conducted by heating or refluxing liquid reactants in a reactor in the presence of a drying agent and a small amount of catalyst in the absence of air or oxygen using a gentle constant nitrogen or other inert gas purge.

### EXAMPLE A

A 250 mL three neck round bottom flask was fit with a condenser, a thermometer and a nitrogen inlet tube and heated by a thermally controlled heating mantle. It was supplied with 10 grams of lime, 75 grams of ethanol and approximately 0.1 gram of Co₂(C₆H₅O₂)₄ catalyst. A slow nitrogen flow was established, the heating rate set to gentle reflux and the condenser maintained at ice temperature. After two hours of heating the reaction was terminated, the flask allowed to cool to room temperature and products transferred to a sample bottle. Composition was determined by GC analysis of the liquid resulting in formation of 72% ethyl butyl ethers and 7% ethyl hexyl ether products leaving 21% of un-reacted ethanol.

### EXAMPLE B

A 250 mL three neck round bottom flask was fit with a condenser, a thermometer and a nitrogen inlet tube and heated by a thermally controlled heating mantle. It was supplied with 10 grams of lime, 95 grams of ethanol and approximately 0.1 gram of V₂(O₂CCH₃)₄ catalyst. A slow nitrogen flow was established, the heating rate set to gentle reflux and the condenser maintained at ice temperature. After two hours of heating the reaction was terminated, the flask allowed to cool to room temperature and products transferred to a sample bottle. Composition was determined by GC analysis of the liquid resulting in formation of 78% ethyl butyl ethers (increased n- to iso- ratio), 5% ethyl hexyl ether, 1% other products and returning 16% ethanol.

### EXAMPLE C

A 250 mL three neck round bottom flask was fit with a condenser, a thermometer and a nitrogen inlet tube and heated by a thermally controlled heating mantle. It was supplied with 11 grams of lime, 92 grams of n-propanol and approximately 0.1 gram of Cr₂(O₂CCH₃)₄ catalyst. A slow nitrogen flow was established, the heating rate set to gentle reflux and the condenser maintained at ice temperature. After three and one quarter hours of heating the reaction was terminated, the reactor allowed to cool to room temperature and products transferred to a sample bottle. Composition was determined by GC analysis of the liquid resulting in 5% hexanol, 84% propyl hexyl ethers, 9% dihexyl ethers and 2% other products.

### EXAMPLE D

A 250 mL three neck round bottom flask was fit with a thermocouple, a vapor vent tube and a nitrogen inlet tube and was heated by a thermally controlled heating mantle. It was supplied with 120 mL of propylene glycol, approximately 11 grams of lime and 0.07 gram of Co(O₂CCH₃)₂ hydrate catalyst. A slow nitrogen flow was established, the heating rate set to hold the reactant at 180° C. After four hours of heating the reaction was terminated, the flask allowed to cool to room temperature and products transferred to a sample bottle. Composition was estimated by FTIR analysis of the liquid resulting in glycol ethers and unsaturated alcohol products.

### EXAMPLE E

A 250 mL three neck round bottom flask was fit with a thermocouple, a vapor vent tube and a nitrogen inlet tube and was heated by a thermally controlled heating mantle. It was supplied with 36.5 grams of n-butylamine dissolved in 23.0 grams of ethanol, 0.7 gram of Co(O₂CCH₃)₂ hydrate catalyst and 36 grams of a calcium sulfate drying agent. A slow nitrogen flow was established, the heating rate set to hold the reactant at 60° C. After five hours of heating the reaction was terminated, the flask allowed to cool to room temperature and products transferred to a sample bottle. Composition was estimated by evaporative reduction and FTIR analysis of the liquid resulting in approximately 50% ethyl butyl amines, di-butyl amines, butyl hexyl amines and related amine products.

### EXAMPLE F

A 125 mL conical flask was fit with a nitrogen inlet tube and was heated by a thermally controlled hot plate. It was supplied with 12.22 grams of 2,6-dimethylphenol dissolved in 4.61 grams of ethanol, 0.24 gram of Co(O₂CCH₃)₂ catalyst and 3.6 grams of a calcium sulfate drying agent. A slow nitrogen flow was established, the heating rate set to hold the reactant at 60° C. Most of the reactants were lost by vaporization during the heating period. After five hours of heating the reaction was terminated, the flask allowed to cool to room temperature and liquid products transferred to a sample bottle. The products were isolated by evaporation resulting in a brown viscous liquid. Composition was determined by FTIR analysis resulting in less than ten percent of ethylphenyl ether.

### EXAMPLE G

A 125 mL conical flask was fit with a nitrogen inlet tube and was heated by a thermally controlled hot plate. It was supplied with 23.0 grams of ethanol, 29.0 grams of acetone, 0.32 gram of Co(O₂CCH₃)₂ catalyst on calcium sulfate and 30 grams of a calcium sulfate drying agent. A slow nitrogen flow was established, the heating rate set to hold the mixed liquid reactants at ˜45° C. After two hours of heating the reaction was terminated, the flask allowed to cool to room temperature and products transferred to a sample bottle. The products were isolated by evaporating off residual reactants leaving liquid products. Composition of the liquid was determined by FTIR analysis showing production of methyl propyl ketone and allyl ethyl ether.

What is claimed:

1. Ambient pressure non-oxidative catalytic chemical combination of a polar organic reactant in a liquid range comprising an alcohol, glycol, ether, aldehyde, ketone, carboxylic acid, ester, amine, imine, thiol, sulfide, phosphine with another organic reactant comprising an alcohol, glycol, ether, alkane, alkene, aldehyde, ketone, carboxylic acid, ester, amine, imine, thiol, sulfide, phosphine compound possessing at least one active hydrogen atom forming products in the absence of air comprising higher molecular weight alcohol, glycol, ether, alkane, alkene, aldehyde, ketone, carboxylic acid, ester, amine, imine, thiol, sulfide, phosphine or related organic compound by condensation.

2. Ambient pressure non-oxidative catalytic chemical combination of a polar organic reactant in a liquid range comprising an alcohol, glycol, ether, aldehyde, ketone, carboxylic acid, ester, amine, imine, thiol, sulfide, phosphine with another organic reactant comprising an alcohol, glycol, ether, alkane, alkene, aldehyde, ketone, carboxylic acid, ester, amine, imine, thiol, sulfide, phosphine compound possessing at least one active hydrogen atom forming products in the absence of air comprising higher molecular weight alcohol, glycol, ether, alkane, alkene, aldehyde, ketone, carboxylic acid, ester, amine, imine, thiol, sulfide, phosphine or related organic compound by condensation wherein catalysts, possessing a degree of symmetry, are formed in the absence of air from transition metal compounds comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof.

3. Liquid reactants comprising alcohols, glycols, ethers, aldehydes, ketones, carboxylic acids, esters, amines, imines, thiols, sulfides or phosphines are catalytically converted to higher molecular weight products at ambient pressure in the absence of air. Specifically, catalytic reactions are taught for non-oxidative chemical conversion of liquid alcohols to higher boiling alcohols, ethers, glycol ethers, amines and oxygenated gasoline products, comprising ethanol to butanol, propanols to hexanols, butanols to octanols, ethanol plus butanol to ethyl butyl ethers, ethanol plus ethyl butyl ethers to ethyl hexyl ethers as oxygenated gasoline, ethanol plus ethyl hexyl ethers to butyl hexyl ethers as oxygenated gasoline, n-butyl amine plus ethanol to ethyl butyl amine, hexyl amines and related products at reflux temperatures.

4. Liquid reactants comprising alcohols, glycols, ethers, aldehydes, ketones, carboxylic acids, esters, amines, imines, thiols, sulfides or phosphines are catalytically converted to higher molecular weight products at ambient pressure in the absence of air. Specifically, catalytic reactions are taught for non-oxidative chemical conversion of liquid alcohols to higher boiling alcohols, ethers, glycol ethers, amines and oxygenated gasoline products, comprising ethanol to butanol, propanols to hexanols, butanols to octanols, ethanol plus butanol to ethyl butyl ethers, ethanol plus ethyl butyl ethers to ethyl hexyl ethers as oxygenated gasoline, ethanol plus ethyl hexyl ethers to butyl hexyl ethers as oxygenated gasoline, n-butyl amine plus ethanol to ethyl butyl amine, hexyl amines and related products at reflux temperatures wherein catalysts, possessing a degree of symmetry, are formed in the absence of air from transition metal compounds comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof.
